(19) United States
(12) Patent Application Publication
(10) Pub. No.: US 2008/0027325 A1
(43) Pub. Date: Jan. 31, 2008

(54) Ultrasound Apparatus

(75) Inventors: Mark Duncan Brooks, Northcote (AU); Andrew Bruce Moorhouse, Coburn (AU); Anthony Edward Samir, Cambridge, MA (US); Paul Lykhun U, Forest Hill (AU)

Correspondence Address: FITCH EVEN TABIN AND FLANNERY, 120 SOUTH LA SALLE STREET, SUITE 1600, CHICAGO, IL, 60603-3406, US

(73) Assignee: Austin Health, Heidelberg Victoria (AU)

(21) Appl. No.: 10/571,171

(22) PCT Filed: Sep. 10, 2004
(86) PCT No.: PCT/AU04/01239

§ 371 Date: Aug. 28, 2007

(30) Foreign Application Priority Data
Sep. 12, 2003 (AU) 2003904973

(51) Int. Cl. A61B 8/00

(52) U.S. Cl. 600/459

(57) ABSTRACT

There is disclosed an ultrasound apparatus comprising an ultrasound transducer that operates in a target plane, and a light source that emits a broad, planar light beam that is co-planar with said target plane and directed relative to said ultrasound transducer to illuminate at least a region where an instrument is to be aligned with said target plane.

### FIELD OF THE INVENTION

The present invention relates to an ultrasound apparatus and method of assisting in alignment of an instrument relative to an ultrasound transducer probe. One application of the invention is to assist the puncturing of internal body organs, vessels and the like, through the utilization of a puncturing cannula or hollow needle that will reflect ultrasound waves.

### DISCUSSION OF THE PRIOR ART

It is presently known that one can remove tissues or body fluids from internal body organs for example, the liver or kidney, for diagnostic purposes by means of suitable puncturing needles. By the same method amniotic fluid may be removed from the uterus during a pregnancy or, for example, blood or a medication may be injected into the fetal body or the organs of an adult human.

In all of these instances it is extremely important to know the precise position of the puncturing cannula or needle relative to the organs or vessels that are to be punctured so as to avoid any unnecessary injuries of endangered areas (for example, the heart during puncture of the left lobe of the liver), and also to prevent a tissue withdrawal from an erroneous body region or a misplaced injection.

An ultrasound-echo sectional view apparatus having an ultrasonic transducer probe for the ultrasonic scanning of the body region which is to be punctured, and a display for viewing the ultrasound echo-section images, allows continuous puncturing control through the assistance of ultrasound, in particular, through rapid display ultrasound-section images. The ultrasound transducer probe can be adjusted while observing the display of the ultrasound-echo sectional view apparatus to select, in the body region which is to be punctured, a sectional plane that is preferred for the puncture target or aim direction. Once the target direction is chosen, this is displayed as an echo-sectional view. If the puncturing cannula or needle is inserted in the plane of the ultrasound beam it is also easily visible on the display, since the cannula material has a distinguishable ultrasound contrast to the surrounding biological tissue.

Notwithstanding good visual control in the scanning region there are, however, further aiming problems. The movement of the cannula in the tissue may be directly followed by eye on the display only when the cannula actually reaches into the region of the ultrasound-scanning waves in the scanning sectional plane. If the plane of insertion of the cannula is different to that of the scanning sectional plane then the entire needle will not be seen. If the entire needle is not seen then there is a risk that the incorrect organ or tissue may be punctured, with a higher risk of unwanted injury to the patient or removal of incorrect tissue. A factor that potentially exacerbates this problem is that until the needle passes through any subcutaneous fat, the needle can be difficult to observe. Hence, the needle may be significantly misplaced before this is determined.

U.S. Pat. No. 4,058,114 describes a guide that is attached to the ultrasonic transducer probe. The needle is inserted through the guide, and the guide constrains the pathway of the needles such that it remains in the plane of the ultrasound beam.

Such guides limit the ability of the operator to angle the needle independently of the ultrasound transducer probe.

Accordingly, if the needle is inserted at the wrong angle or it is necessary to negotiate an obstacle such as a rib, the constraints of the guide make it difficult or impossible to realign the needle. This necessitates withdrawal of the needle and reinsertion and can cause additional and undesirable trauma to the patient.

### SUMMARY OF THE INVENTION

The invention provides an ultrasound apparatus comprising:
an ultrasound transducer that operates in a target plane; and
a light source that emits a broad, planar light beam that is co-planar with said target plane and directed relative to said ultrasound transducer to illuminate at least a region where an instrument is to be aligned with said target plane.

The invention also provides a method of assisting in alignment of an instrument relative to an ultrasound transducer that operates in a target plane comprising directing a broad, planar light beam in the same plane as said target plane to illuminate at least a region where said instrument is to be aligned.

The invention provides a method of aligning an instrument relative to an ultrasound transducer that operates in a target plane comprising directing a broad planar light beam in the same plane as said target plane and to illuminate at least a region where said instrument is to be aligned; and
adjusting the position of said instrument by monitoring light from said light source reflected from said instrument to determine whether said instrument is in said target plane.

Embodiments of the invention facilitate the insertion of a puncturing cannula into the body region that is to be punctured, in the same plane as the ultrasound beam, while allowing the operator to angle the needle as desired during the procedure without moving the ultrasonic transducer probe.

### BRIEF DESCRIPTION OF THE DRAWINGS

Further advantages and details of the invention may be ascertained from the following description of embodiments thereof, taken in conjunction with the accompanying drawings in which:

FIG. 1 is a schematic diagram of an ultrasound apparatus being used to assist in a puncturing procedure;

FIG. 2 is a schematic diagram illustrating the ultrasound transducer probe of the first embodiment; and

FIG. 3 is a schematic diagram illustrating the ultrasound transducer probe of a second embodiment.

### DETAILED DESCRIPTION

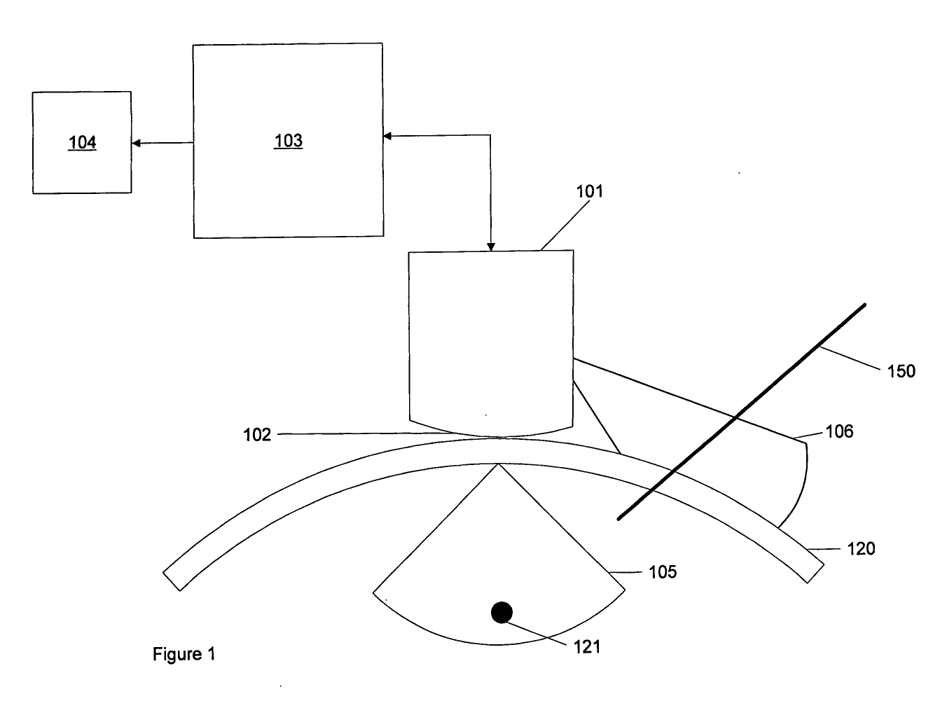

FIG. 1 illustrates schematically how an ultrasound apparatus 100 of a first embodiment can be used to align an instrument with the target plane.

Herein, the term “instrument” is used to refer to any item that may be desired to be monitored or guided using an ultrasound including puncturing cannulae, needles and the like.

The term “target plane” is used to refer to the plane in which the ultrasound operates—i.e., the plane from which the ultrasound transducer receives reflected sound waves that are subsequently processed and displayed.

The apparatus 100 comprises a transducer probe 101 that generates and receives sound waves by means of piezoelectric crystals. As is well known in the art, by applying appropriate electric currents to the crystals, sound waves are produces which travel outward from the crystals. Reflected sound waves are transformed by the piezoelectric crystals into electric current. The processor/controller 103 of the ultrasound apparatus converts these electric currents into ultrasound images as is well known to persons skilled in the art. The ultrasound images are then displayed on display 104. Processor/controller 103 also contains control means for controlling the ultrasound transducer which is typically mounted at the contact end 102 of the ultrasound transducer probe 101.

A coupling medium, for example, a precedent water section, is applied on the skin surface of a patient in the elevation of a target organ that is to be punctured, for example, the liver or the uterus of a pregnant woman to couple the transducer to the skin. The ultrasound beam is radiated in the direction of the target organ and reflected back to the ultrasound scanning probe. The reflected ultrasound beam 105, thereby scans this body region and, in particular, the target organ 121 that is to be punctured—i.e. the transducer probe 101 is adjusted until the target organ 121 is displayed.

Through the corresponding linewise reproduction of the ultrasound echo impulses emanating from each ultrasound line in the examination region, on the display 104 there is obtained a visual image of the target plane of the target organ, that has been presently scanned by the ultrasonic beam. In order to assist in the insertion of an instrument along the plane of the ultrasound beam the ultrasound transducer probe incorporates a light source in the form of a laser assembly that emits a laser beam 106. The laser beam is a broad, planar laser beam 106. The laser is mounted so that the plane of the emitted light is co-planar with the target plane.

To successfully intersect the target 121, the needle is inserted in the plane of the laser beam and is thereby colinear with the target plane. The needle will thus be visible on the display as it lies in the plane of the ultrasound beam when it is within the patient. The operator can monitor light reflected from the needle to align the needle appropriately, i.e. the longer the line of reflected light, the closer the needle is to the correct plane. This is particularly advantageous where the needle is being inserted into a body that has a layer of subcutaneous fat as the needle or other instrument can be difficult to observe in the region of subcutaneous fat and therefore will not appear on the ultrasound until it has been displaced some distance into the body. Accordingly, if the needle is offline, without the guidelight of the transducer probe of the present embodiment, the operator will not expect to see the needle until sometime after the needle has been inserted, and accordingly, an operator can be tempted to continue to insert the needle further into the body in situations where the needle is not visible because it is in the wrong plane rather than it is obscured by subcutaneous fat. Using the apparatus and inserting a needle in accordance with the aid of the apparatus of the preferred embodiment, allows the operator a greater degree of certainty that the needle will appear in the target plane while maintaining flexibility for the operator to adjust the needle position. This allows the operator to negotiate obstacles —for example, a bone such as a rib.

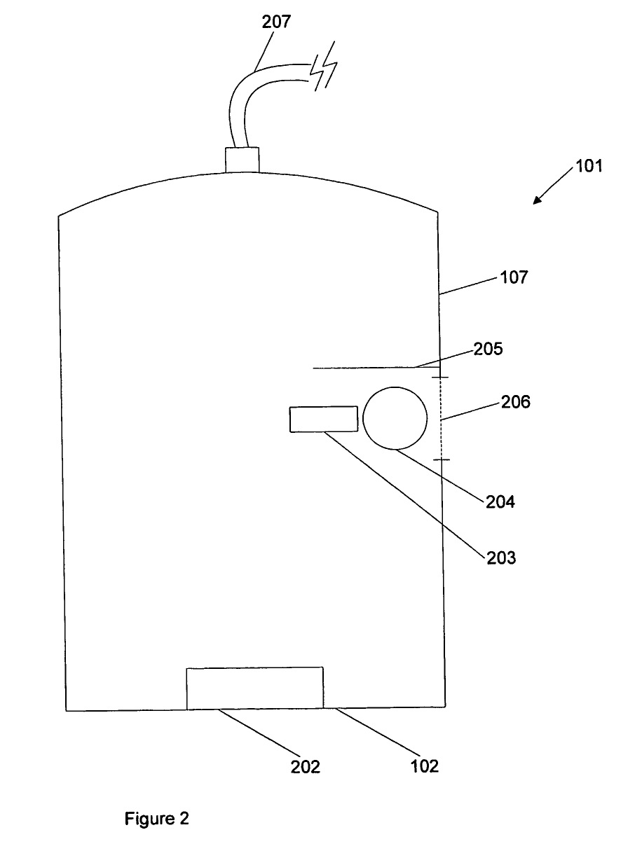

Further details of an ultrasound probe 101 of a first preferred embodiment are illustrated in FIG. 2. In FIG. 2 the ultrasound transducer probe 101 is connected by cable 207 to processor/controller 103. The ultrasound transducer 202 is mounted in the contact end 102 of the ultrasound transducer 101 probe. The control circuitry for the transducer 202 is well known to persons skilled in the art and is accordingly not illustrated.

The laser 203 is mounted within casing 107. The laser 203 is also turned on or off under operation of the controller 103.

Cylindrical lens 204 is mounted within the casing and turns the linear light beam produced by laser 203 to a broad planar light beam. In order to conveniently direct as much light as possible to the region where the instrument is to be aligned, mirror 205 is placed above window 206. Thus, light is emitted from window 206 to a region near the ultrasound device in order to enable alignment of an instrument.

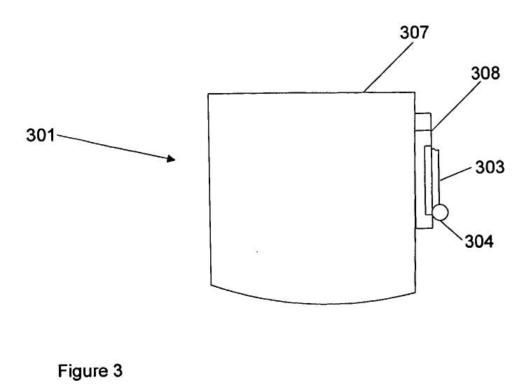

A second embodiment of the invention is shown in FIG. 3 where a laser assembly consisting of a laser module 308, a laser 303 and a cylindrical lens 304 are mounted externally to the casing 307 of an ultrasound transducer probe. The laser assembly may be permanently or demountably mounted to the probe. The laser module, incorporates a power source and switch for turning laser 303 on or off. In all other respects, the apparatus operates as in the first embodiment. While making the laser assembly demountable offers certain advantages, it would also be appreciated that mounting the light source within the casing of the transducer probe provides the advantage that the transducer probe is otherwise shaped as conventional probes. This is convenient in terms of supply of disposable covers which can be used to keep the transducer probe sterile.

While there has been shown what is considered to be the preferred embodiment of the invention, it will be obvious that modifications may be made which come within the scope of the disclosure of the specification.

For example, while a laser light source is convenient it will be appreciated that other light sources could be used such as light emitting diodes with appropriate focussing optics. These and other modifications should be understood as falling within the scope of the invention.

1. An ultrasound apparatus comprising:
an ultrasound transducer that operates in a target plane, said ultrasound transducer mounted in a casing; and
a light source mounted within said casing that emits a broad, planar light beam that is co-planer with said target plane, said casing further comprising a window through which said light beam is emitted, said light beam being directed relative to said ultrasound transducer to illuminate at least a region where an instrument is to be aligned with said target plane, and wherein said light source comprises a laser and two optical elements, a first optical element for transforming light from said laser into a planar beam and a second optical element for directing light to the region where the instrument is to be aligned.

2-6. (canceled)

7. An ultrasound apparatus as claimed in claim 1, wherein said first optical element comprises a cylindrical lens.

8. An ultrasound device as claimed in claim 1, wherein said second optical element comprises a mirror.

9. An ultrasound apparatus as claimed in claim 1, further comprising:
a processor for processing signals from said ultrasound transducer to produce ultrasound images; and
a display for displaying said ultrasound images.

10-12. (canceled)
